(19) United States
(12) Patent Application Publication
(10) Pub. No.: US 2026/0258188 A1
(43) Pub. Date: Sep. 3, 2026

(54) BISMUTH-CONTAINING CATALYST COMPRISING A DICARBOXYLATE LIGAND

(71) Applicant: BASF SE, Ludwigshafen am Rhein (DE)

(72) Inventors: Emre LEVENT, Ludwigshafen am Rhein (DE); Peter DEGLMANN, Ludwigshafen am Rhein (DE); Oliver SALA, Ludwigshafen am Rhein (DE); Oliver WELZ, Ludwigshafen am Rhein (DE); Peter HOFFMANN, Muenster (DE)

(73) Assignee: BASF SE, Ludwigshafen am Rhein (DE)

(21) Appl. No.: 18/878,348

(22) PCT Filed: Jun. 22, 2023
(86) PCT No.: PCT/EP2023/067015

§ 371 Date: Dec. 23, 2024

(30) Foreign Application Priority Data
Jun. 24, 2022 (EP) 22181006.2

(51) Int. Cl. C08G 18/22; C07C 57/38; C08G 59/68; C08G 63/08; C08G 63/82

(52) U.S. Cl. CPC C08G 18/227; C07C 57/38; C08G 59/685; C08G 63/08; C08G 63/823

(57) ABSTRACT

The invention relates to a bismuth-containing catalyst as such, which is defined by the general formula (I) detailed in the subsequent text. The bismuth-containing catalyst comprises as a dicarboxylate ligand a dianion (R¹)²— according to the general formula (II), wherein the dianion (R¹)²⁻ comprises the radicals R³, R⁴, R⁵ and R⁶ in α-position. The present invention further relates to a method for preparing the bismuth-containing catalyst of this kind and to the use of such a bismuth-containing catalyst in reactions for preparing compounds comprising a urethane group, as an esterification and transesterification catalyst and as a catalyst for ring-opening polymerizations of lactones and epoxides.

The invention relates to a bismuth-containing catalyst as such, which is defined by the general formula (I) detailed in the subsequent text. The bismuth-containing catalyst comprises as a dicarboxylate ligand a dianion (R¹)²⁻ according to the general formula (II), wherein the dianion (R¹)²⁻ comprises the radicals R³, R⁴, R⁵ and R⁶ in α-position. The present invention further relates to a method for preparing the bismuth-containing catalyst of this kind and to the use of such a bismuth-containing catalyst in reactions for preparing compounds comprising a urethane group, as an esterification and transesterification catalyst and as a catalyst for ring-opening polymerizations of lactones and epoxides.

WO 2018/069018 relates to a coating composition system comprising the components (A) to (C) and optionally further components. The component (A) is at least one polyhydroxyl group-containing compound and the component (B) is at least one polyisocyanate-containing compound. In contrast, the component (C) is a catalyst comprising at least two salts of an aliphatic monocarboxylic acid having at least 4 carbon atoms. In this case, the metal component of the first salt is bismuth (Bi), while the second salt comprises magnesium (Mg), sodium (Na), potassium (K) or calcium (Ca) as metal component. The coating composition system according to WO 2018/069018 may be configured according to a first option such that all components are present separately from one another, i.e. the individual components are not mixed with one another, whereas according to a second option of the corresponding coating composition system, the respective components can also be present completely or at least partially mixed with one another.

U.S. Pat. No. 4,895,827 discloses a catalyst in the form of a metal salt, in which the catalyst is a constituent of a heat-sensitive colour-forming composition, which, in addition to the catalyst, comprises a chromogenic material comprising an acidic developer and a suitable binder. The metal salt may comprise different metals as central metal atom/metal ion comprising, for example, zinc, tin, aluminum or nickel. The corresponding metal salt comprises organic compounds as ligands, which, in addition to a carboxyl group, also comprise aromatic fragments and vinyl groups. However, bismuth-containing catalysts are not disclosed in U.S. Pat. No. 4,895,827.

JP-A 58 87 087 discloses the use of a multivalent metal salt of Diphenyl acetic acid, especially Diphenyl zinc acetate, as colorants, wherein a water-proof coloured picture can be produced. However, bismuth-containing metal salts or the use of metal salts for preparing a compound comprising a urethane bond are not disclosed in JP-A 58 87 087.

WO 2020/160939 relates to a bismuth-containing catalyst comprising at least one radical R¹, which comprises a carboxyl fragment, wherein a first carbon atom (α-carbon) is bonded to the carbon atom of the carboxyl group, which in turn is directly substituted with at least one aromatic system. WO 2020/160939 further relates to a method for preparing the bismuth-containing catalyst and to the use of the bismuth-containing catalyst for preparing compounds comprising a urethane group.

The preparation of compounds comprising a urethane group (urethane bond) has likewise been known for a long time. A compound having a urethane group is generally obtained if a compound comprising an isocyanate group is reacted with a compound comprising a hydroxyl group. The reaction generally takes place in the presence of a catalyst. Although tin-containing catalysts exhibit very high activity in such reactions, the use of such tin-containing catalysts, especially alkyl-tin compounds, should be avoided owing to their (very high) toxicity.

The object of the present invention, therefore, was to provide a novel catalyst, which can be used for preparing compounds comprising a urethane group.

This object is achieved by a bismuth-containing catalyst of a general formula (I)

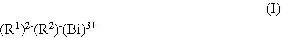

in which the variables are defined as follows:
(R¹)²⁻ is mutually independently a dianion of a general formula (II)

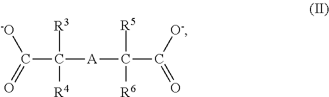

wherein R³, R⁴, R⁵ and R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆ -C₁₄-aryl and the alkyl and aryl fragments of these substituents may in turn be at least monosubstituted by hydroxyl, halogen, —CF₃, —NH₂, —SH, or C₁-C₆-alkoxy,
and wherein A is unsubstituted or at least monosubstituted C₁-C₃₀-alkylene, wherein A may comprise a heteroatom selected from the group consisting of O, S, P—R¹² and N—R¹²,
wherein R¹² is unsubstituted or at least monosubstituted C₁-C₃₀-alkyl or H, wherein R¹² may comprise a heteroatom selected from the group consisting of O, S, P—R¹³ and N—R¹³, wherein R¹³ is an unsubstituted C₁-C₃₀-alkyl or H,
(R²)⁻ is mutually independently a hydroxide anion, an alkoxylate anion, a halide anion, a hydrogencarbonate anion, a thiolate anion, R⁷, an anion of a general formula (III)

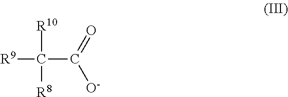

or an anion of a general formula (IV)

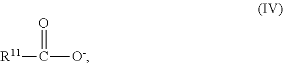

wherein R⁷ is unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein R⁸ and R⁹ are each phenyl and R¹⁰ is C₁-C₁₂-alkyl,
and wherein R¹¹ is unsubstituted or at least monosubstituted C₁-C₃₀-alkyl, C₆-C₁₄-aryl or C₇-C₃₀-aralkyl,
wherein the substituents are selected from hydroxyl, halogen, carboxyl, —CF₃, —NH₂, —SR¹⁴, C₁-C₆-alkoxy, C₁-C₃₀-alkyl or C₆-C₁₄-aryl, wherein the alkyl and aryl fragments of these substituents may in turn be at least monosubstituted by hydroxyl, halogen, —CF₃, —NH₂ or C₁-C₆-alkoxy and R¹⁴ is H, unsubstituted or at least monosubstituted C₁-C₃₀-alkyl, C₆-C₁₄-aryl or C₇-C₃₀-aralkyl.

The bismuth-containing catalysts according to the invention are characterized in that, inter alia, the use of toxic tin-containing catalysts in the production of compounds comprising a urethane group can be avoided. The bismuth-containing catalysts according to the invention have a comparable catalytic activity as known representatives of the effective, on the one hand catalytically active, but on the other hand toxic, tin-containing catalysts.

However, the catalytic activity of the bismuth-containing catalysts according to the invention is better than the corresponding catalytic activity of the bismuth- or zinc-containing catalysts already known.

Moreover, the bismuth-containing catalysts according to the invention exhibit improved hydrolytic stability compared to bismuth-containing catalysts already known. For example, low amounts of water can already be sufficient in order to significantly or fully annihilate the catalytic activity of bismuth-containing catalysts based on purely aliphatic monocarboxylate ligands, such as laurate-containing bismuth catalysts. Whereas such catalysts known from the prior art start to precipitate on contact with water, the bismuth-containing catalysts according to the invention are much more stable. Owing to the increased stability to hydrolysis—and thus storage stability—the bismuth-containing catalysts according to the invention exhibit their improved catalytic properties over a much longer time period.

Furthermore, in the case of the bismuth-containing catalysts according to the invention, it is also not required that the catalyst as a salt is employed in the presence of protonated ligand. The bismuth-containing catalysts according to the invention can thus be used without the presence of the corresponding acid at high catalytic activity in order to form compounds having urethane groups and there are no issues how the corresponding acid as a low molecular species behaves in the respective formulation and later in the polymeric material.

Advantageous properties are then already obtained in the catalysts according to the invention if the dianion (R¹)²⁻ according to the general formula (II), which is used as substituent/ligand of the bismuth central atom, comprises the radicals R³, R⁴, R⁵ and R⁶ in α-position and the diradical A. It is preferred that at least one of the radicals R³, R⁴, R⁵ or R⁶ is unsubstituted or at least monosubstituted C₆-C₁₄-aryl, especially phenyl.

“α-position” in the context of the present invention describes the carbon atom next to the carbonyl carbon atom of the carboxylic acid. In accordance with the invention, this carbon atom is referred to as the α-carbon. Known examples for this purpose from chemical nomenclature are α-amino acids, where the α-C atom is the carbon atom to which the amino group and the carboxyl group are attached. Specific examples for this numbering from the field of amino acids are β-alanine and gamma-aminobutyric acid. In chemical nomenclature, the carbonyl carbon is sometimes also counted and referred to as position 1. Accordingly, said first carbon atom directly adjacent to the carbon atom of the carboxyl group is sometimes also referred to as position 2 in chemical nomenclature. In the context of the present invention, the dianion (R¹)²⁻ according to the general formula (II) has two α-carbons.

The said carboxyl groups of this substituent are located (spatially speaking) in proximity to the bismuth central atom of the bismuth-containing catalyst. The bismuth-containing catalysts according to the invention are represented as salts, wherein the bismuth central atom of the bismuth-containing catalyst according to the invention is represented as a (triple positively charged) cation of the corresponding salt (see for example the general formula (I)). The corresponding substituents/ligands of the bismuth-containing catalyst, which are represented by the substituents/radicals R¹ and R² in the general formula (I) detailed above, form the corresponding anion components of the bismuth-containing catalyst in this salt representation. The substituent/ligand R¹ is double negatively charged and the substituent/ligand R² is singly negatively charged. As detailed below, the substituent R¹ comprises two carboxyl groups and the substituent R² can comprise one carboxyl group. In general, the negative charge in the corresponding substituents/ligands of said carboxyl groups is localized and/or the corresponding carboxyl groups are located in spatial proximity to the (positively charged) bismuth central atom.

From a scientific standpoint however, it is also tenable, in place of the salt notation used in the context of the present application for the bismuth-containing catalysts according to the invention, to select a notation/representation in which chemical bonds between the bismuth central atoms and the ligands R¹ and R² according to general formula (I) is completely or at least partially formed in each case. Expressed in other words, this means that the bismuth central atom is not present as a positively charged cation and the corresponding ligands are also not present as negatively charged anions, but rather the corresponding charge form a chemical bond between the corresponding ligands on the one hand and the bismuth central atom on the other hand. In the context of the present invention, the bismuth-containing catalysts disclosed according to the invention therefore also describe such a definition that is not based on a salt.

In the context of the present invention, definitions such as C₁-C₃₀-alkyl, such as defined, for example, for the radicals R³ to R⁶ in formula (II) above, signifies that this substituent (radical) is an alkyl radical having a carbon atom number of 1 to 30, wherein substituents optionally present are not taken into consideration in the carbon atom number. The alkyl radical may be either linear or branched as well as optionally cyclic. Alkyl radicals having both a cyclic and a linear component also fall under this definition. The same applies to other alkyl radicals such as a C₁-C₆-alkyl radical or a C₁-C₁₂-alkyl radical for example. Examples of alkyl radicals are methyl, ethyl, n-propyl, sec-propyl, n-butyl, sec-butyl, isobutyl, 2-ethylhexyl, tertiary-butyl (tert-Bu/t-Bu), pentyl, hexyl, heptyl, cyclohexyl, octyl, nonyl or decyl.

In the context of the present invention, definitions such as C₁-C₃₀-alkylene, such as defined, for example, for the diradical A in formula (II) above, signifies that this diradical is an alkylene diradical having a carbon atom number of 1 to 30, wherein substituents optionally present are not taken into consideration in the carbon atom number. The alkyl radical may be either linear or branched.

In the context of the present invention, a heteroatom signifies any atom that is not a carbon or a hydrogen atom and that has replaced a carbon atom in the backbone of the molecular structure of a compound, especially in the C₁-C₃₀-alkylene bridge of formula (II). Examples of heteroatoms are O, S, P and N.

In the context of the present invention, the term “aryl” or the term “C₆-C₁₄-aryl”, as defined, for example, for the radicals R³ to R⁶ in formula (II) above, signifies that the substituent (radical) is an aromatic system. The corresponding aromatic system has a carbon atom number of 6 to 14, wherein substituents optionally present are not taken into consideration in the carbon atom number. The aromatic system may be a monocyclic, bicyclic or optionally polycyclic aromatic system. In the case of bicyclic or polycyclic aromatic systems, individual rings may optionally be fully or partially saturated. Preferably, all rings of the corresponding aromatic systems are fully unsaturated. Preferred examples of aryl are phenyl, naphthyl or anthracyl, especially phenyl.

In the context of the present invention, the definition “C₇-C₃₀-aralkyl”, as defined for example for the radicals R³ to R⁶ in formula (II) above, signifies that the substituent (radical) comprises an alkyl radical (such as C₁-C₆-alkyl according to the definitions above), wherein this alkyl radical is in turn substituted by an aryl radical (according to the definitions above). The corresponding aralkyl substituent has a carbon atom number of 7 to 30, wherein substituents optionally present are not taken into consideration in the carbon atom number. The alkyl radical itself present therein may be either linear or branched as well as optionally cyclic.

In the context of the present invention, the term “C₁-C₆-alkoxy”, as defined for example as (additional) substituent of the radicals R³ to R⁶ in formula (II) above, signifies that it is a substituent (radical) in this case which is derived from an alcohol. The corresponding substituent thus comprises an oxygen fragment (—O—), which is in turn linked to an alkyl radical, such as C₁-C₆-alkyl (according to the definitions above). The alkyl radical itself may be either linear or branched as well as optionally cyclic.

In the context of the present invention, the term “halogen”, as defined for example as (additional) substituent of the radicals R³ to R⁶ in formula (II) above, signifies that the substituent (radical) is fluorine, chlorine, bromine or iodine, preferably fluorine or chlorine, particularly preferably chlorine.

In the context of the present invention, the term “unsubstituted or at least monosubstituted C₁-C₃₀-alkyl, C₆-C₁₄-aryl or C₇-C₃₀-aralkyl”, such as defined for example for the radicals R³ to R⁶ in formula (II) above, signifies that each of the in total four substituents (radicals) detailed corresponding to their definitions already specified above may be present either in unsubstituted form or have at least one further substituent (monosubstituted). If one or more substituents are present (for example disubstituted, trisubstituted or even higher substituted), the appropriate substituents are selected independently of one another from the substituent groups specified in each case.

In the case of a disubstituted C₆-C₁₄-aryl for example, the corresponding aryl unit, such as phenyl for example, may be substituted for example by a hydroxyl and a C₁-C₃₀-alkyl substituent, such as methyl or ethyl. Alkyl or aryl fragments may themselves in turn comprise at least one additional substituent according to the definitions stated. The substitution may be at any desired position of the corresponding fragment.

Unless otherwise specified in the following description, the respective definitions of the radicals R¹ to R¹¹ are in each case the preferred unsubstituted definitions.

The present invention is further specified herein below.

The present invention firstly relates to a bismuth-containing catalyst of the general formula (I)

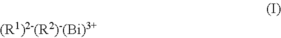

in which the variables are defined as follows:
(R¹)²⁻ is mutually independently a dianion of a general formula (II)

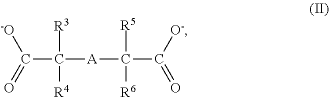

wherein R³, R⁴, R⁵ and R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein A is unsubstituted or at least monosubstituted C₁-C₃₀-alkylene, wherein A may comprise a heteroatom selected from the group consisting of O, S, P—R¹² and N—R¹²,
wherein R¹² is unsubstituted or at least monosubstituted C₁-C₃₀-alkyl or H, wherein R¹² may comprise a heteroatom selected from the group consisting of O, S, P—R¹³ and N—R¹³, wherein R¹³ is an unsubstituted C₁-C₃₀-alkyl or H,
(R²)⁻ is mutually independently a hydroxide anion, an alkoxylate anion, a halide anion, a hydrogencarbonate anion, a thiolate anion, R⁷, an anion of a general formula (III)

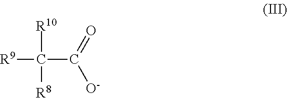

or an anion of a general formula (IV)

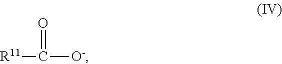

wherein R⁷ is unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein R⁸ and R⁹ are each phenyl and R¹⁰ is C₁-C₁₂-alkyl, and wherein R¹¹ is unsubstituted or at least monosubstituted C₁-C₃₀-alkyl, C₆-C₁₄-aryl or C₇-C₃₀-aralkyl,
wherein the substituents are selected from hydroxyl, halogen, carboxyl, CF₃, —NH₂, —SR¹⁴, C₁-C₆-alkoxy, C₁-C₃₀-alkyl or C₆-C₁₄-aryl, wherein the alkyl and aryl fragments of these substituents may in turn be at least monosubstituted by hydroxyl, halogen, —CF₃, —NH₂ or C₁-C₆-alkoxy and R¹⁴ is H, unsubstituted or at least monosubstituted C₁-C₃₀-alkyl, C₆-C₁₄-aryl or C₇-C₃₀-aralkyl.

In connection with the radicals (substituents/ligands) present in the general formula (I), particularly the necessary radicals R¹ and R², it should be noted that the further/exact chemical definition of these radicals R¹ or R² is a result of the radicals R³ to R⁶, A, R¹² and R¹³ of the general formula (II) with respect to the radical R¹ and is a result of the radicals R⁸ to R¹¹ of the general formulae (III) or (IV) and of the radicals R⁷ and R¹⁴ with respect to the radical R².

### R¹

The dianion of the general formula (II) is preferably defined according to the invention such that at least one of the radicals R³, R⁴, R⁵ or R⁶ is unsubstituted or at least monosubstituted C₆-C₁₄-aryl, more preferably at least two, most preferably at least three, and especially preferably each of the radicals R³, R⁴, R⁵ or R⁶ are unsubstituted or at least monosubstituted C₆-C₁₄-aryl.

Another object of the present invention is, therefore, a bismuth-containing catalyst in which at least one of the radicals R³, R⁴, R⁵ or R⁶ is unsubstituted or at least monosubstituted C₆-C₁₄-aryl, preferably at least two, more preferably at least three, and most preferably each of the radicals R³, R⁴, R⁵ or R⁶ are unsubstituted or at least monosubstituted C₆-C₁₄-aryl.

In a preferred embodiment, at least one, preferably at least two, more preferably at least three, and most preferably each of the radicals R³, R⁴, R⁵ or R⁶ is phenyl.

Another object of the present invention is, therefore, a bismuth-containing catalyst in which at least one, preferably at least two, more preferably at least three, and most preferably each of the radicals R³, R⁴, R⁵ or R⁶ is phenyl.

In one preferred embodiment, A is unsubstituted C₁-C₃₀-alkylene, preferably unsubstituted C₃-C₁₂-alkylene.

Thus, another object of the present invention is a bismuth-containing catalyst in which A is unsubstituted C₁-C₃₀-alkylene, preferably unsubstituted C₃-C₁₂-alkylene.

In another preferred embodiment, A is unsubstituted C₁-C₃₀-alkylene and comprises a heteroatom selected from the group consisting of O, S, P—R¹² and N—R¹², preferably selected from the group consisting of O, S and N—R¹², more preferably selected from O and S, wherein R¹² is unsubstituted or at least monosubstituted C₁-C₃₀-alkyl or H, wherein R¹² may comprise a heteroatom selected from the group consisting of O, S, P—R¹³ and N—R¹³, wherein R¹³ is an unsubstituted C₁-C₃₀-alkyl or H.

Thus, another object of the present invention is a bismuth-containing catalyst in which A is unsubstituted C₁-C₃₀-alkylene and comprises a heteroatom selected from the group consisting of O, S, P—R¹² and N—R¹², preferably selected from the group consisting of O, S and N—R¹², more preferably selected from O and S, wherein R¹² is unsubstituted or at least monosubstituted C₁-C₃₀-alkyl or H, wherein R¹² may comprise a heteroatom selected from the group consisting of O, S, P—R¹³ and N—R¹³, wherein R¹³ is an unsubstituted C₁-C₃₀-alkyl or H.

In this case, it is further preferred that A is unsubstituted C₃-C₁₂-alkylene and comprises a heteroatom selected from the group consisting of O, S, P—R¹² and N—R¹², preferably selected from the group consisting of O, S and N—R¹², more preferably selected from O and S,
wherein R¹² is unsubstituted C₃-C₅-alkyl or H, wherein R¹² may comprise a heteroatom selected from the group consisting of O, S, P—R¹³ and N—R¹³, wherein R¹³ is a methyl.

Thus, another object of the present invention is a bismuth-containing catalyst in which A is unsubstituted C₃-C₁₂-alkylene and comprises a heteroatom selected from the group consisting of O, S, P—R¹² and N—R¹², preferably selected from the group consisting of O, S and N—R¹², more preferably selected from O and S,
wherein R¹² is unsubstituted C₃-C₅-alkyl or H, wherein R¹² may comprise a heteroatom selected from the group consisting of O, S, P—R¹³ and N—R¹³, wherein R¹³ is a methyl.

The corresponding dicarboxylic acid of the dianion (R¹)²⁻ of a general formula (IIa)

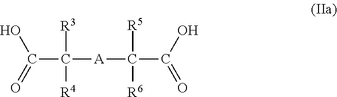

preferably has a negative ΔG value upon its exchanging for two monocarboxylate ligands, for example pivalate, computationally determined by ab-initio or semi-empirical methods, e.g. at the DFT (density functional theory) level of theory.

Another object of the present invention is therefore a bismuth-containing catalyst in which the corresponding dicarboxylic acid of the dianion (R¹)²⁻ of a general formula (IIa)

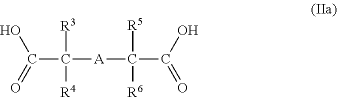

has a negative ΔG value upon its exchanging for two monocarboxylate ligands, computationally determined by ab-initio or semi-empirical methods.

Examples of suitable corresponding dicarboxylic acids of the dianion (R¹)²⁻ of a general formula (IIa) are shown in table 1. The ΔG values upon their exchanging for two monocarboxylate ligands (pivalate; see reaction equation in the first line of table 1) are computationally determined by DFT (density functional theory). The DFT calculations are carried out with the program package TURBOMOLE (version 7.5.2) at the M06-2X/def2-TZVPD//BP86/def-TZVP/COSMO (ε=∞) level of theory, employing COSMO-RS solvation (BP_TZVPD_FINE_C30_1801.ctd parameterization, 1:1 mixture of Ethylhexyl-O—C(═O)—NH—C₆H₁₂—NCO and EtOH at 25° C.). Negative values of ΔG for the below ligand exchange reaction indicate that chelates are favored. Thus, the more negative these values are for the dicarboxylic acids, the more stable the expected chelate will be, from which higher (hydrolytic) stability can be concluded. Based on these data, conclusions on optimal linker sizes and additional donor atoms can be drawn.

TABLE 1

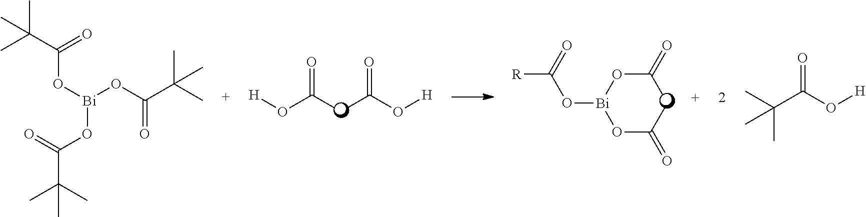

| 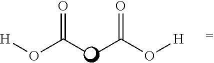 | ΔG [kJ/mol] (M06-2X) |
|---|---|
| 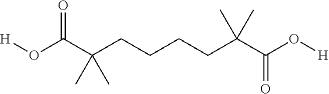 [SMILES: CC(C)(CCCCC(C)(C)C(=O)O)C(=O)O] | −6.5 |
| 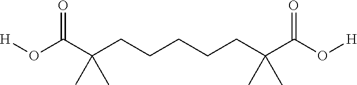 [SMILES: CC(C)(CCCCCC(C)(C)C(=O)O)C(=O)O] | −13.6 |
| 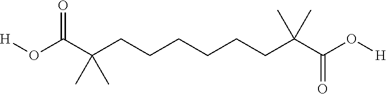 [SMILES: CC(C)(CCCCCCC(C)(C)C(=O)O)C(=O)O] | −16.1 |
| 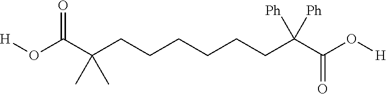 [SMILES: CC(C)(CCCCCCC(C(=O)O)(c1ccccc1)c1ccccc1)C(=O)O] | −13.4 |
| 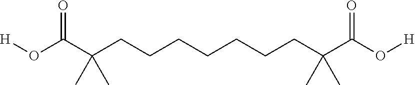 [SMILES: CC(C)(CCCCCCCC(C)(C)C(=O)O)C(=O)O] | −12.2 |
| 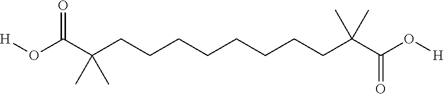 [SMILES: CC(C)(CCCCCCCCC(C)(C)C(=O)O)C(=O)O] | −3.3 |

| | |
|---|---|
| 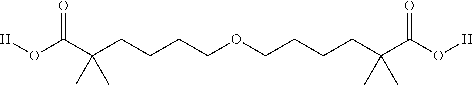 [SMILES: CC(C)(CCCCOCCCCC(C)(C)C(=O)O)C(=O)O] | −9.6 |
| 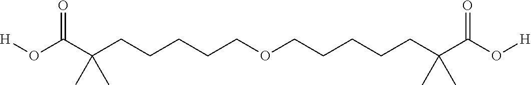 [SMILES: CC(C)(CCCCCOCCCCCC(C)(C)C(=O)O)C(=O)O] | −12.5 |
| 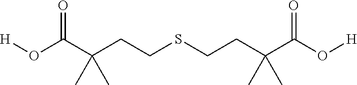 [SMILES: CC(C)(CCSCCC(C)(C)C(=O)O)C(=O)O] | −12.7 |
| 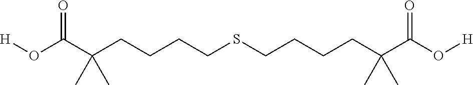 [SMILES: CC(C)(CCCCSCCCCC(C)(C)C(=O)O)C(=O)O] | −23.4 |
| 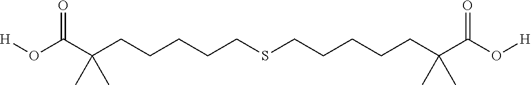 [SMILES: CC(C)(CCCCCSCCCCCC(C)(C)C(=O)O)C(=O)O] | −15.7 |
| 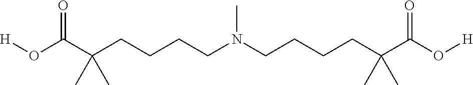 [SMILES: CN(CCCCC(C)(C)C(=O)O)CCCCC(C)(C)C(=O)O] | −30.7 |
| 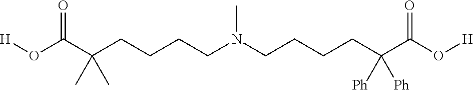 [SMILES: CN(CCCCC(C)(C)C(=O)O)CCCCC(C(=O)O)(c1ccccc1)c1ccccc1] | −24.8 |
| 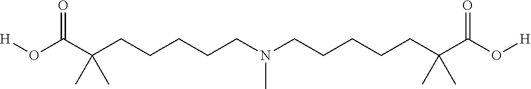 [SMILES: CN(CCCCCC(C)(C)C(=O)O)CCCCCC(C)(C)C(=O)O] | −17.4 |
| 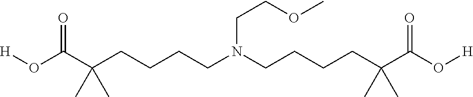 [SMILES: COCCN(CCCCC(C)(C)C(=O)O)CCCCC(C)(C)C(=O)O] | −35.9 |
| 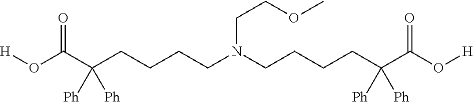 [SMILES: COCCN(CCCCC(C(=O)O)(c1ccccc1)c1ccccc1)CCCCC(C(=O)O)(c1ccccc1)c1ccccc1] | −34.5 |
| 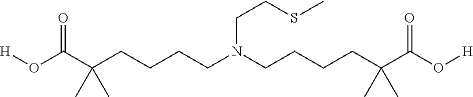 [SMILES: CSCCN(CCCCC(C)(C)C(=O)O)CCCCC(C)(C)C(=O)O] | −19.1² |
| 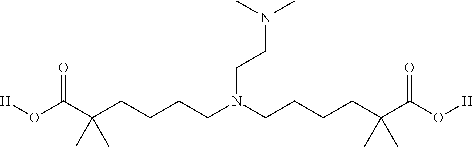 [SMILES: CN(C)CCN(CCCCC(C)(C)C(=O)O)CCCCC(C)(C)C(=O)O] | −46.3 |
| 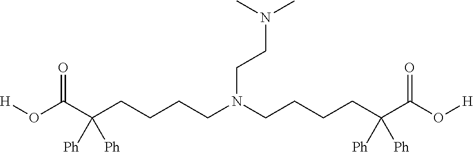 [SMILES: CN(C)CCN(CCCCC(C(=O)O)(c1ccccc1)c1ccccc1)CCCCC(C(=O)O)(c1ccccc1)c1ccccc1] | −24.2 |
| 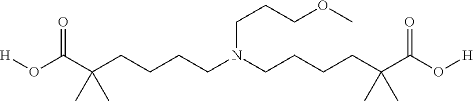 [SMILES: COCCCN(CCCCC(C)(C)C(=O)O)CCCCC(C)(C)C(=O)O] | −31.7 |
| 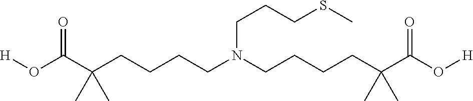 [SMILES: CSCCCN(CCCCC(C)(C)C(=O)O)CCCCC(C)(C)C(=O)O] | −13.0² |

²Value for most stable conformer with thioetheric S coordinated to Bi

### R²

Preferably, (R²)⁻ is mutually independently a hydroxide anion, an alkoxylate anion, a thiolate anion, a chloride anion, R⁷, a radical of the general formula (III) or a radical of the general formula (IV), wherein R⁷ is unsubstituted or at least monosubstituted C₁-C₁₂-alkyl or C₆-C₁₄-aryl, wherein the substituents are selected from the group consisting of hydroxyl, chlorine, —CF₃ and C₁-C₆-alkyl.

Another object of the present invention is therefore a bismuth-containing catalyst in which (R²)⁻ is mutually independently a hydroxide anion, an alkoxylate anion, a thiolate anion, a chloride anion, R⁷, a radical of the general formula (III) or a radical of the general formula (IV), wherein R⁷ is unsubstituted or at least monosubstituted C₁-C₁₂-alkyl or C₆-C₁₄-aryl, wherein the substituents are selected from the group consisting of hydroxyl, chlorine, —CF₃ and C₁-C₆-alkyl.

In case (R²)⁻ is an anion of the general formula (III), R⁸ and R⁹ are preferably each phenyl and R¹⁰ is C₆-C₁₀-alkyl, especially C₈-alkyl.

The present invention further relates also to a method for preparing a bismuth-containing catalyst of the general formula (I) according to the definitions above. The method according to the invention for preparing such bismuth-containing catalysts can comprise, for example, reacting
i) at least one compound of a general formula (IIa)

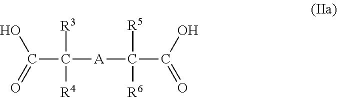

or a corresponding salt thereof,
wherein R³, R⁴, R⁵ und R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein A is unsubstituted or at least monosubstituted C₁-C₃₀-alkylene, wherein A may comprise a heteroatom selected from the group consisting of O, S, P—R¹² and N—R¹²,
wherein R¹² is unsubstituted or at least monosubstituted C₁-C₃₀-alkyl or H, wherein R¹² may comprise a heteroatom selected from the group consisting of O, S, P—R¹³ and N—R¹³, wherein R¹³ is an unsubstituted C₁-C₃₀-alkyl or H,
ii) optionally at least one compound of a general formula (IIIa)

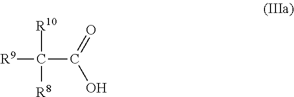

or a corresponding salt thereof,
wherein R⁸ and R⁹ are each phenyl and R¹⁰ is C₁-C₁₂-alkyl, or
at least one compound of a general formula (IVa)

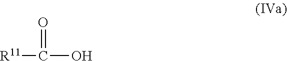

or a corresponding salt thereof,
wherein R¹¹ is unsubstituted or at least monosubstituted C₁-C₃₀-alkyl, C₆-C₁₄-aryl or C₇-C₃₀-aralkyl, wherein the substituents are selected from hydroxyl, halogen, carboxyl, —CF₃, —NH₂, —SR¹⁴, C₁-C₆-alkoxy, C₁-C₃₀-alkyl or C₆-C₁₄-aryl, wherein the alkyl and aryl fragments of these substituents may in turn be at least monosubstituted by hydroxyl, halogen, —CF₃, —NH₂ or C₁-C₆-alkoxy and R¹⁴ is H, unsubstituted or at least monosubstituted C₁-C₃₀-alkyl, C₆-C₁₄-aryl or C₇-C₃₀-aralkyl, and
iii) at least one bismuth-containing compound selected from the group consisting of Bi₂O₃, bismuth carbonate, bismuth hydrogencarbonate, bismuth halide, bismuth carboxylates, bismuth alkoxylates, Bi(C₆-C₁₄-aryl) 3, Bi(C₁-C₁₂-alkyl) 3 and metallic bismuth.

The reactants listed above, i.e. the acids according to the general formulae (IIa) to (IVa) or the appropriate corresponding salts as such, are known to those skilled in the art. The corresponding salts used can be, for example, sodium, potassium or calcium salts. Optionally, instead of the aforementioned acids according to the general formulae (Ila) to (IVa) or corresponding salts thereof as reactants, it is also possible to use corresponding carboxylic esters, for example a methyl or ethyl ester. Such carboxylic esters can be prepared by reacting the aforementioned acids or a corresponding salt thereof with a suitable alcohol, for example methanol or ethanol, optionally in the presence of a catalyst. The appropriate preparation methods of such carboxylic esters are known to a person skilled in the art.

In principle, any bismuth-containing compound can be used in the method according to the invention, which is suitable for the purpose of forming the bismuth central atom in the bismuth-containing catalyst of the general formula (I) according to the invention, by reaction with the appropriate compounds according to the general formulae (IIa) to (IVa). Bismuth-containing compounds as such are known to those skilled in the art. If, in accordance with the invention, a bismuth halide is used as bismuth-containing compound, it is preferably a chlorine-containing compound, especially BiCl₃. Any specific substituents/substitution patterns, such as the radicals R⁸ to R¹⁰ for example, may already be present in the corresponding reactant. Optionally, such substituents/substitution patterns can also be attached or completed even after the preparation process of a bismuth-containing catalyst according to the general formula (I) described above.

Preferably, the bismuth-containing compound is selected from the group consisting of Bi₂O₃, BiCl₃, bismuth alkoxides, bismuth carboxylates, Bi(C₆H₅)₃ and metallic bismuth.

In a preferred embodiment, the bismuth-containing catalysts according to the general formula (I) according to the invention are prepared by reacting at least one compound of the general formula (IIa) and at least one compound of the general formulae (IIIa) or (IVa) with at least one bismuth-containing compound, wherein
i) the reaction is carried out under a protective atmosphere and/or in the presence of at least one solvent, preferably toluene or tetrahydrofuran, and/or
ii) the reaction is conducted for at least 3 hours and/or at a temperature in the range of −75 to 160° C., and/or
iii) following the reaction, volatile constituents are removed, the bismuth-containing catalyst is dried under reduced pressure and/or a recrystallization is carried out.

Furthermore, as mentioned above, the at least one compound of the general formula (IIa) preferably has a negative ΔG value upon its exchanging for two monocarboxylate ligands, computationally determined by ab-initio or semi-empirical methods.

The at least one bismuth-containing catalyst according to the definitions above can be used in Lewis-acid catalysed reactions, for example, in esterifications, transesterifications, ring-opening polymerizations of ethers, lactones, epoxides and amines, epoxidations and in reactions for preparing compounds comprising a urethane group, preferably in reactions for preparing compounds comprising a urethane group.

The present invention therefore further relates to the use of at least one bismuth-containing catalyst according to the definitions above in reactions for preparing compounds comprising a urethane group.

The present invention therefore further relates to the use of at least one bismuth-containing catalyst according to the definitions above as an esterification and transesterification catalyst.

The present invention therefore further relates to the use of at least one bismuth-containing catalyst according to the definitions above as a catalyst for ring-opening polymerizations of lactones and epoxides.

The invention is illustrated hereinafter by examples.

### EXAMPLES

### Preparation of Inventive and Comparative Catalysts

### I) Preparation of Precursors

### Ia) Precursor 1:2,2,9,9-Tetraphenyldecanedioic acid (Compound of a General Formula (IIa); tpddH)

2,2,9,9-Tetraphenyldecanedioic acid was prepared using a modified literature procedure (C. Destabel, J. D. Kilburn, J. Knight, Tetrahedron 1994, 50, 11267).

In a Schlenk flask, 2,2-diphenylacetic acid (50.0 g, 235.57 mmol, 1.00 eq.) was dissolved in 156 mL THF (tetrahydrofuran) and cooled to −15° C. Then, a 2.5 M solution of n-butyllithium in n-hexane (210 mL, 525 mmol, 2.20 eq.) was slowly added and the resulting red solution was stirred for 45 min at this temperature. Subsequently, the reaction solution was cooled to −45° C. and 1,6-dibromohexane (28.73 g, 117.79 mmol, 0.50 eq.) was slowly added. The mixture was then stirred overnight at room temperature and terminated by the addition of a 1.0 M aqueous solution of HCl. During acidification, a solid precipitate was obtained, which was then washed with water and dried overnight at 80° C. in vacuo to afford the dicarboxylic acid.

Yield: 98% (58.20 g, 115 mmol)

Characterization by 1H-NMR, 13C-NMR, HRMS (High-resolution mass spectrometry) and infrared spectroscopy.

### Ib) Precursor 2:6,6′-oxybis(2,2-diphenylhexanoic acid) (Compound of a General Formula (IIa); O-bis(dph)H)

6,6′-oxybis(2,2-diphenylhexanoic acid) was prepared using a modified literature procedure.

In a Schlenk flask, 2,2-diphenylacetic acid (50.0 g, 235.57 mmol, 1.00 eq.) was dissolved in 156 mL THF and cooled to −15° C. Then, a 2.5 M solution of n-butyllithium in n-hexane (210 mL, 525 mmol, 2.20 eq.) was slowly added and the resulting red solution was stirred for 45 min at this temperature. Subsequently, the reaction solution was cooled to −45° C. and bis(4-chlorobutyl) ether (21.70 mL, 117.79 mmol, 0.50 eq.) was slowly added. The mixture was then stirred overnight at room temperature and terminated by the addition of a 1.0 M aqueous solution of HCl. Subsequently, the phases were separated in a separatory funnel and the aqueous phase was extracted with Et₂O (3×50 mL). Afterwards, the collected organic phases were washed with water, dried over MgSO₄ and decanted. Crystallization at −27° C. gives a white solid.

Yield: 75% (48.16 g, 87.45 mmol)

Characterization by 1H-NMR, 13C-NMR, HRMS (High-resolution mass spectrometry) and infrared spectroscopy.

### Ic) Precursor 3:2,2-Diphenyldecanoic acid (Compound of a General Formula (IIIa); dpdH)

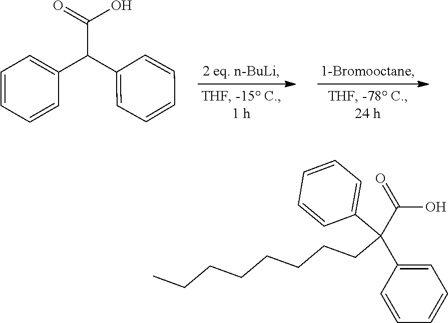

2,2-Diphenylacetic acid (10.6 g; 48 mmol) were dissolved in 75 mL of dry tetrahydrofuran (THF) under a protective gas atmosphere (argon or nitrogen) in a 200 mL Schlenk flask and cooled to −15° C. A 1.6M solution of n-butyllithium in hexane (60 mL; 96 mmol) was then added with stirring and over a period of 30 minutes. The reaction solution was stirred at −15° C. for one hour and cooled to −78° C. for the addition of 1-bromooctane (8.3 mL; 48 mmol). Subsequently, the reaction solution was slowly warmed to room temperature and stirred for a further 24 hours.

For the work-up and purification of the 2,2-diphenyldecanoic acid, a saturated ammonium chloride solution (60 mL) was added to the reaction solution and stirred for 30 minutes. The aqueous phase was separated by means of a separating funnel and extracted with 3×25 mL of diethyl ether. The combined organic phases were dried over magnesium sulfate (MgSO₄). All volatile solvents were then removed under reduced pressure (1·10⁻³ mbar) and the resulting solid dried at 140° C. under reduced pressure (1·10⁻³ mbar) for 24 hours.

Characterization by 1H-NMR, 13C-NMR, HRMS (high-resolution mass spectrometry), infra-red spectroscopy.

### II) General Procedure for the Preparation of the Inventive Catalysts

The inventive bismuth (III) carboxylates according to the general formula (I) were prepared following a modified synthetic procedure of Levent et al (E. Levent, O. Sala, L. F. B. Wilm, P. Loewe, F. Dielmann, Green Chem., 2021, 23, 2747).

As monocarboxylic acids 2,2-diphenyldecanoic acid (dpdH; compound of a general formula (IIIa)), neodecanoic acid (ndaH; compound of a general formula (IVa)), 2,2-diphenylpropionic acid (dppH; commercially available from Sigma-Aldrich; compound of a general formula (IIIa)), lauric acid (IauH; compound of a general formula (IVa)) and 2-((phenylthio)methyl)benzoic acid (SPhbzaH; commercially available from Sigma-Aldrich; compound of a general formula (IVa)) were used.

A Schlenk flask was charged with BiPh₃ (1.00 eq.), dicarboxylic acid (1.00 eq.; compound of a general formula (IIa)) and a monocarboxylic acid (1.00 eq.). Anhydrous toluene or tetrahydrofuran (3 mL per mmol of BiPh₃) was added and the reaction mixture was heated for at least 16 hours at 110° C. The complete consumption of BiPh₃ was determined by 1H-NMR monitoring of the reaction mixture. Subsequently, all volatiles were removed under reduced pressure and the residue was dried in vacuo at 80° C. overnight to afford the bismuth (III) carboxylate as an off-white solid.

Characterization by 1H-NMR, 13C-NMR, HRMS (High-resolution mass spectrometry) and infrared spectroscopy.

The prepared inventive catalysts are shown in table 2.

TABLE 2

| Inventive | | |
|---|---|---|
| Examples | (R¹)²⁻ | (R²)⁻ |
| I1 | tpdd | dpd |
| I2 | tpdd | nda |
| I3 | tpdd | lau |
| I4 | tpdd | dpp |
| I5 | tpdd | SPhbza |
| I6 | O-bis(dph) | dpd |

tpdd, dpd and so on correspond to the respective anion of the (di) carboxylic acids tpddH, dpdH and so on.

### III) Preparation of the Comparative Catalysts

### IIIa) Comparative Example C1: Bi(2,2-diphenyldecanoate)₃ (Bi(dpd)₃)

Triphenylbismuth (1.1 g; 2.5 mmol) and 2,2-diphenyldecanoic acid (2.43 g; 7.5 mmol) were initially charged under a protective gas atmosphere in a 25 mL three-necked flask equipped with stirrer bar, reflux condenser, thermometer and protective gas atmosphere inlet (argon or nitrogen). 12.5 mL of dry tetrahydrofuran or dry toluene (5 mL of solvent per 1 mmol of triphenylbismuth) were added to the reactants and the mixture is heated at 110° C. under a protective gas atmosphere for at least 16 hours. The reaction course was monitored by 1H-NMR (nuclear magnetic resonance spectroscopy). After complete conversion of triphenylbismuth with formation of benzene, the reaction was terminated and cooled. All volatile solvents were then removed under reduced pressure (1·10⁻³ mbar) and the resulting solid dried at 60° C. under reduced pressure (1·10⁻³ mbar) for 24 hours. As required, the resulting compound was purified from toluene and hexane at −40° C. or by recrystallization from hot toluene.

Characterization by 1H-NMR, 13C-NMR, C/H/N elemental analysis, infra-red spectroscopy.

### IIIb) Comparative Example C2: Bi(nda)₃

nda is neodecanoate (commercially available catalyst from Sigma Aldrich); in neodecanoic acid having a metal content of 21% by weight

### IV) Determination of the Catalytic Activity of the Catalysts

The respective catalytic activity of the individual inventive and comparative examples was tested by means of a reaction in which a compound is formed comprising a urethane group, wherein the NCO conversion was measured with a IR spectrometer (FT-IR Spectrometer 7000 e, Agilent; the measuring range was between 500 to 4000 cm⁻¹).

The catalytic crosslinking reaction between an NCO group (of a polyisocyanate) and an OH group (of an OH-functional polyacrylate) to form a polyurethane is investigated by means of heatable horizontal ATR-IR spectroscopy at 60° C. The measurements were made directly after addition of the curing agent solution to the mixing varnish. The starting point was measured at ambient temperature with a formulation without catalyst. The reaction was subsequently monitored in 3-minute steps over a time of 30 minutes at 60° C. The conversion was determined from the decrease in the intensity of the band for the free isocyanate at 2260 cm⁻¹. The spectra were standardized by reference to the isocyanurate band at 1690 cm⁻¹.

As polyisocyanate (item 6; curing agent) a mixture of 95 parts of HMDI trimer (NCO content 23.5±0.5%) with 5 parts of an IPDI trimer (NCO content 11.9±0.4%) diluted to a solids content of 85% in a 1:1 mixture of butyl acetate and xylene was used.

As OH-functional polyacrylate (item 1), a polyacrylate polyol having an OH number of 150 mg KOH/g (based on solid content) and an acid number in the range from 1 to 15 mg KOH/g (based on solid content) was used. The following monomers were used in the production of the polyacrylate polyol: Methyl methacrylate, n-butyl acrylate, butyl methacrylate, hydroxypropyl methacrylate.

As diluent (item 7) a 1:1 mixture of xylene/butyl acetate (solvent) was used.

The composition of the mixing varnish is shown in table 3. The figures for the concentrations are absolute amounts (expressed in parts), based on the overall mixing varnish (Exception: Catalyst).

TABLE 3

| | | |
|---|---|---|
| 1 | Polyacrylate polyol | 74 |
| 2 | Varnish additives | 13.6 |
| 3 | Light stabilizer | 2.1 |
| 4 | Ethylethoxypropionate | 8.6 |
| 5 | Catalyst | The Bi |
| | | content was |
| | | 0.2 mmol. |
| 6 | Polyisocyanate (Curing agent) | 33 |
| 7 | Diluent | 33 |

To produce the mixing varnish, items 1 to 4 of table 3 were combined with stirring. The corresponding quantities of catalyst were added to the mixing varnish and mixed. The curing agent (item 6) and the diluent (item 7) were added immediately before the paint-specific values to be determined experimentally (IR measurement).

For testing the water stability (in tables 4 and 5: Example+H₂O), items 1 to 4 of table 3 were combined with stirring. The corresponding quantities of catalyst were mixed with 1.0% by weight (based on overall weight of reactants) of water and the diluent and the mixture left to stand for 10 minutes. Then, the mixture was added to the mixing varnish. The curing agent (item 6) was added immediately before the paint-specific values to be determined experimentally (IR measurement).

The results are shown in tables 4 (inventive examples) and 5 (comparative examples).

TABLE 4

| | I1 | I1 + H₂O | I2 | I2 + H₂O | I3 | I3 + H₂O |
|---|---|---|---|---|---|---|
| | NCO | NCO | NCO | NCO | NCO | NCO |
| t | degradation | degradation | degradation | degradation | degradation | degradation |
| [min] | [%] | [%] | [%] | [%] | [%] | [%] |
| 0 | 0 | 0 | 0 | 0 | 0 | 0 |
| 3 | 40.4 | 36.5 | 38.3 | 33.1 | 36.6 | 26.8 |
| 6 | 57.2 | 54.5 | 54.8 | 51.1 | 53.7 | 43.8 |
| 9 | 64.9 | 62.7 | 63.8 | 60.6 | 62.9 | 54.1 |
| 12 | 69.5 | 67.6 | 69 | 66.5 | 68.7 | 61.1 |
| 15 | 72.4 | 70.7 | 72.3 | 70.5 | 72.6 | 65.7 |
| 18 | 75 | 72.8 | 75.2 | 73.3 | 75.5 | 69.2 |
| 21 | 76.7 | 74.5 | 77.1 | 75.5 | 77.6 | 72 |
| 24 | 78.3 | 75.9 | 78.5 | 77.1 | 79.2 | 74.2 |
| 27 | 79.6 | 77.1 | 80 | 78.5 | 80.5 | 76 |
| 30 | 80.7 | 77.9 | 81 | 79.7 | 81.6 | 77.5 |
| | I4 | I4 + H₂O | I5 | I5 + H₂O | I6 | I6 + H₂O |
| | NCO | NCO | NCO | NCO | NCO | NCO |
| t | degradation | degradation | degradation | degradation | degradation | degradation |
| [min] | [%] | [%] | [%] | [%] | [%] | [%] |
| 0 | 0 | 0 | 0 | 0 | 0.0 | 0.0 |
| 3 | 34.4 | 32.2 | 24.1 | 22.2 | 48.0 | 41.8 |
| 6 | 51.5 | 49.8 | 38.7 | 37.3 | 64.6 | 58.5w |
| 9 | 61.5 | 59.4 | 48.4 | 47.3 | 72.4 | 67.5 |
| 12 | 67.9 | 65.2 | 54.8 | 54.6 | 76.9 | 72.7 |
| 15 | 72 | 68.9 | 60 | 59.8 | 79.8 | 76.3 |
| 18 | 75.3 | 71.9 | 63.5 | 63.7 | 81.9 | 78.8 |
| 21 | 77.7 | 73.9 | 66.5 | 66.8 | 83.7 | 80.8 |
| 24 | 79.5 | 75.6 | 69.0 | 69 | 84.9 | 82.3 |
| 27 | 80.9 | 76.8 | 71.3 | 71 | 86.0 | 83.7 |
| 30 | 82.5 | 78.2 | 73.1 | 73.1 | 86.8 | 84.7 |

TABLE 5

| | C1 | C1 + H₂O | C2 | C2 + H₂O |
|---|---|---|---|---|
| | NCO | NCO | NCO | NCO |
| t | degradation | degradation | degradation | degradation |
| [min] | [%] | [%] | [%] | [%] |
| 0 | 0 | 0 | 0 | 0 |
| 3 | 30.8 | 21.7 | 27.1 | 17.4 |
| 6 | 48.8 | 39.2 | 46.2 | 29.5 |
| 9 | 59 | 50.2 | 55.4 | 38 |
| 12 | 65.5 | 57.4 | 61.2 | 44.6 |
| 15 | 69.7 | 61.7 | 65.4 | 49.7 |
| 18 | 72.9 | 65.1 | 68.4 | 53.5 |
| 21 | 75.2 | 67.7 | 70.6 | 57.2 |
| 24 | 76.9 | 69.9 | 72.7 | 60 |
| 27 | 78.3 | 71.6 | 74.6 | 62.5 |
| 30 | 79.6 | 73 | 75.9 | 64.5 |

As can be deduced from tables 4 and 5, the catalysts according to the invention show, according to inventive examples 11 to 16, a comparable or higher, respectively, catalytic activity compared to the known catalysts according to comparative examples C1 and C2. After, the addition of water, the catalysts according to the invention show, according to inventive examples 11 to 16, a significantly increased catalytic activity compared to the known catalysts according to comparative examples C1 and C2 which shows their strongly improved hydrolytic stability. The improved hydrolytic stability of the examples I6 to I6 according to the invention compared to the comparative examples C1 and C2 should be particularly emphasized here, as determined based on the percentage reactivity loss due to the addition of water (the comparative examples C1 and C2 exhibit the greatest reactivity loss due to the addition of water).

1. A bismuth-containing catalyst of a general formula (I) 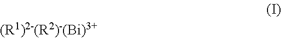
in which the variables are defined as follows:
(R¹)²⁻ is mutually independently a dianion of a general formula (II) 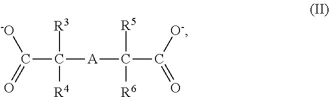
wherein R³, R⁴, R⁵ and R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein A is unsubstituted or at least monosubstituted C₁-C₃₀-alkylene, wherein A may comprise a heteroatom selected from the group consisting of O, S, P—R¹² and N—R¹²,
wherein R¹² is unsubstituted or at least monosubstituted C₁-C₃₀-alkyl or H, wherein R¹² may comprise a heteroatom selected from the group consisting of O, S, P—R¹³ and N—R¹³, wherein R¹³ is an unsubstituted C₁-C₃₀-alkyl or H,
(R²) is mutually independently a hydroxide anion, an alkoxylate anion, a halide anion, a hydrogencarbonate anion, a thiolate anion, R⁷, an anion of a general formula (III) 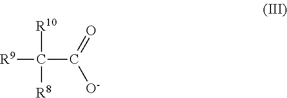
or an anion of a general formula (IV) 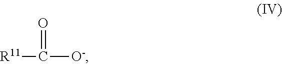
wherein R⁷ is unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein R⁸ and R⁹ are each phenyl and R¹⁰ is C₁-C₁₂-alkyl,
and wherein R¹¹ is unsubstituted or at least monosubstituted C₁-C₃₀-alkyl, C₆-C₁₄-aryl or C₇-C₃₀-aralkyl,
wherein the substituents are selected from hydroxyl, halogen, carboxyl, —CF₃, —NH₂, —SR¹⁴, C₁-C₆-alkoxy, C₁-C₃₀-alkyl or C₆-C₁₄-aryl, wherein the alkyl and aryl fragments of these substituents may in turn be at least monosubstituted by hydroxyl, halogen, —CF₃, —NH₂ or C₁-C₆-alkoxy and R¹⁴ is H, unsubstituted or at least monosubstituted C₁-C₃₀-alkyl, C₆-C₁₄-aryl or C₇-C₃₀-aralkyl.

2. The bismuth-containing catalyst as claimed in claim 1, wherein
the corresponding dicarboxylic acid of the dianion (R¹)²⁻ of a general formula (IIa) 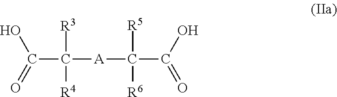
has a negative ΔG value upon its exchanging for two monocarboxylate ligands, computationally determined by ab-initio or semi-empirical methods.

3. The bismuth-containing catalyst as claimed in claim 1, wherein at least one of the radicals R³, R⁴, R⁵ or R⁶ is unsubstituted or at least monosubstituted C₆-C₁₄-aryl.

4. The bismuth-containing catalyst as claimed in claim 1, wherein at least one of the radicals R³, R⁴, R⁵ or R⁶ is phenyl.

5. The bismuth-containing catalyst as claimed in claim 1, wherein A is unsubstituted C₁-C₃₀-alkylene.

6. The bismuth-containing catalyst as claimed in claim 1, wherein A is unsubstituted C₁-C₃₀-alkylene and comprises a heteroatom selected from the group consisting of O, S, P—R¹² and N—R¹²,
wherein R¹² is unsubstituted or at least monosubstituted C₁-C₃₀-alkyl or H,
wherein R¹² may comprise a heteroatom selected from the group consisting of O, S, P—R¹³ and N—R¹³, wherein R¹³ is an unsubstituted C₁-C₃₀-alkyl or H.

7. The bismuth-containing catalyst as claimed in claim 6, wherein A is unsubstituted C₃-C₁₂-alkylene and comprises a heteroatom selected from the group consisting of O, S, P—R¹² and N—R¹²,
wherein R¹² is unsubstituted C₃-C₅-alkyl or H, wherein R¹² may comprise a heteroatom selected from the group consisting of O, S, P—R¹³ and N—R¹³, wherein R¹³ is a methyl.

8. The bismuth-containing catalyst as claimed in claim 1, wherein (R²)⁻ is mutually independently a hydroxide anion, an alkoxylate anion, a thiolate anion, a chloride anion, R⁷, a radical of the general formula (III) or a radical of the general formula (IV),
wherein R⁷ is unsubstituted or at least monosubstituted C₁-C₁₂-alkyl or C₆-C₁₄-aryl, wherein the substituents are selected from the group consisting of hydroxyl, chlorine, —CF₃ and C₁-C₆-alkyl.

9. A method for preparing a bismuth-containing catalyst of the general formula (I) as claimed in claim 1, comprising reacting
i) at least one compound of a general formula (IIa) 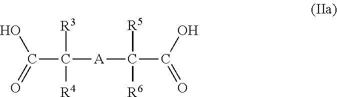
or a corresponding salt thereof,
wherein R³, R⁴, R⁵ und R⁶ are mutually independently unsubstituted or at least monosubstituted C₁-C₃₀-alkyl, C₆-C₁₄-aryl or C₇-C₃₀-aralkyl,
wherein the substituents are selected from the group consisting of hydroxyl, halogen, carboxyl, —CF₃, —NH₂, —SH, C₁-C₆-alkoxy, C₁-C₃₀-alkyl and C₆-C₁₄-aryl and the alkyl and aryl fragments of these substituents may in turn be at least monosubstituted by hydroxyl, halogen, —CF₃, —NH₂, —SH, or C₁-C₆-alkoxy,
and wherein A is unsubstituted or at least monosubstituted C₁-C₃₀-alkylene, wherein A may comprise a heteroatom selected from the group consisting of O, S, P—R¹² and N—R¹²,
wherein R¹² is unsubstituted or at least monosubstituted C₁-C₃₀-alkyl or H, wherein R¹² may comprise a heteroatom selected from the group consisting of O, S, P—R¹³ and N—R¹³ wherein R¹³ is an unsubstituted C₁-C₃₀-alkyl or H,
ii) optionally at least one compound of a general formula (IIIa) 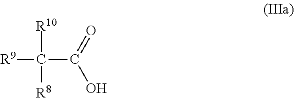
or a corresponding salt thereof,
wherein R⁸ and R⁹ are each phenyl and R¹⁰ is C₁-C₁₂-alkyl, or
at least one compound of a general formula (IVa) 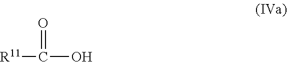
or a corresponding salt thereof,
wherein R¹¹ is unsubstituted or at least monosubstituted C₁-C₃₀-alkyl, C₆-C₁₄-aryl or C₇-C₃₀-aralkyl, wherein the substituents are selected from hydroxyl, halogen, carboxyl, —CF₃, —NH₂, —SR¹⁴, C₁-C₆-alkoxy, C₁-C₃₀-alkyl or C₆-C₁₄-aryl, wherein the alkyl and aryl fragments of these substituents may in turn be at least monosubstituted by hydroxyl, halogen, —CF₃, —NH₂ or C₁-C₆-alkoxy and R¹⁴ is H, unsubstituted or at least monosubstituted C₁-C₃₀-alkyl, C₆-C₁₄-aryl or C₇-C₃₀-aralkyl, and
iii) at least one bismuth-containing compound selected from the group consisting of Bi₂O₃, bismuth carbonate, bismuth hydrogencarbonate, bismuth halide, bismuth carboxylates, bismuth alkoxylates, Bi(C₆-C₁₄-aryl) 3, Bi(C₁-C₁₂-alkyl) 3 and metallic bismuth.

10. The method as claimed in claim 9, wherein the bismuth-containing compound is selected from the group consisting of Bi₂O₃, BiCl₃, bismuth alkoxides, bismuth carboxylates, Bi(C₆H₅)₃ and metallic bismuth.

11. The method as claimed in claim 9, wherein
i) the reaction is carried out under a protective atmosphere and/or in the presence of at least one solvent, and/or
ii) the reaction is conducted for at least 3 hours and/or at a temperature in the range of −75 to 160° C., and/or
iii) following the reaction, volatile constituents are removed, the bismuth-containing catalyst is dried under reduced pressure and/or a recrystallization is carried out.

12. The method as claimed in claim 9, wherein the at least one compound of the general formula (IIa) has a negative ΔG value upon its exchanging for two monocarboxylate ligands, computationally determined by ab-initio or semi-empirical methods.

13. A method for preparing compounds a compound comprising a urethane group, the method comprising performing a reaction in the presence of the bismuth-containing catalyst of claim 1.

14. A method of esterification or transesterification, comprising performing a reaction in the presence of the bismuth-containing catalyst of claim 1.

15. A method of ring-opening polymerization of a lactone or epoxide, comprising performing a reaction in the presence of the bismuth-containing catalyst of claim 1.
